(19) United States
(12) Patent Application Publication
(10) Pub. No.: US 2003/0157499 A1
(43) Pub. Date: Aug. 21, 2003

(54) Method of assessing the amount of nucleic acid in a sample

(76) Inventors: Joakim Lundeberg, Stockholm (SE); Mostafa Ronaghi, Palo Alto, CA (US)

Correspondence Address: Janet M Macleod, Dorsey & Whitney, 250 Park Avenue, New York, NY, 10177, US

(21) Appl. No.: 10/149,842

(22) PCT Filed: Dec. 8, 2000
(86) PCT No.: PCT/GB00/04715

(30) Foreign Application Priority Data
Dec. 10, 1999 (GB) GB 9929381.3

(51) Int. Cl. C12Q 1/68; C12P 19/34

(52) U.S. Cl. 435/6; 435/91.2

(57) ABSTRACT

The present invention provides a method of assessing the amount of target nucleic acid in a sample which comprises co-amplifying the target nucleic acid and at least one competitor nucleic acid molecule wherein each competitor molecule contains a unique discriminatory sequence, and determining the relative amounts of the respective amplicons, characterised in that determination is achieved by detecting a primer extension reaction using each said amplicon as template and a kit for use in such a method.

This invention relates to a method of quantifying nucleic acid and in particular to a competitive assay for nucleic acid which has particular utility in the detection or diagnosis of genetic alterations or infections.

Medical conditions may be diagnosed by the identification of specific target nucleic acid, for example the presence of nucleic acid characteristic of an invading pathogen e.g. viral DNA or RNA and more particularly by monitoring virus copy number. Alternatively, genetic alterations (e.g. mutations) characteristic of or diagnostic for a given medical condition may be detected. Obtaining quantitative data about nucleic acid is useful in analysing the amount of an organism or organisms, e.g. bacteria, in a sample where the quantitative data relating to the nucleic acid is indicative of the number of organisms present in the sample. Also, quantitative information about specific genes, for example by analysis of mRNA, yields useful data about expression patterns of genes of interest. Expression of genes may be up regulated or down regulated in certain medical conditions and such studies are also useful for genomic analyses or environmental monitoring, contamination testing etc.

Target DNA molecules are often present in cell lysates or other source materials in extremely small quantities and in order to amplify such DNA selectively, the polymerase chain reaction (PCR) method has been developed. In this technique a pair of polymerisation primers, specific to known sequences of the target DNA to be amplified, are selected, one primer hybridising at or towards the 5′ end of one of the strands of the target DNA and the other primer at or towards the 5′ end of the second strand, such that in the presence of a polymerase, each primer produces a DNA sequence corresponding to the length of the target DNA template from the terminal of the primer sequence to the other end of the DNA molecule. If the DNA so produced is then subjected to strand separation, typically by melting at a temperature of about 90° C., the newly formed single stranded DNA sequences will hybridise to excess primer present in the mixture, usually after reducing the temperature to the range suitable for annealing, whereupon in the presence of the polymerase, further DNA strands are synthesised, this time extending only between the termini of the two primers. The polymerase is preferably capable of surviving the high temperature used in the strand separation step, thus a thermophilic polymerase, namely Taq DNA polymerase, may advantageously be used. If an excess of the two primers and of nucleotides needed for DNA synthesis is maintained in the medium, it is possible to operate a repeated cyclic process in which the separate strands are synthesised, separated, annealed to primer and new strands synthesised, merely by raising and lowering the temperature between the optimal temperatures for each of the above stages. In this way, it is found that amplification of the original target DNA can be exponential and million-fold increases of concentration can be effected in a relatively short time.

In the detection of bacteria, virus and parasites for example, PCR has several advantages compared to conventional diagnostic methods, i.e. the generality and speed of the assay. However, the fact that conventional PCR assays are only qualitative limits their use to diagnostic applications where only the presence or absence of the pathogen is to be determined. For many diseases, a quantitative measurement is needed to make a proper diagnosis and it would be useful to be able to measure the amount of pathogen during treatment to make a relevant prognosis. As mentioned above, there is major concern regarding the usefulness of PCR assay because their extreme sensitivity makes it possible to obtain false positives as a result of single molecules contaminating the sample. Even small differences in the efficiency of amplification reaction can greatly affect the final accumulation of PCR products. There is therefore a need for a quantitative assay, e.g. one suitable for clinical assays, which overcomes the drawbacks associated with conventional PCR assays.

A number of systems to quantify the initial template DNA/RNA have been described (see for example A. C. Syvänen, M. Bengtström, J. Tenhunen and H. Söderlund Nucl. Acids Res., 16, 11327 (1988); G. Gilliland, S. Perrin and H. F. Bunn PCR protocols, pp. 60-69, Academic Press, San Diego (1990); M. Becker-André and K. Hahlbrock, Nucl. Acids Res. 17,9437 (1990); and N. Kato, O. Yokosuka, K. Hosoda, Y. Ito, M. Okto and M. Omata, Hepatology 18, 16-20 (1993)).

Such methods involve quantization techniques e.g. based on isotope labelling or restriction analysis, which are difficult or cumbersome to operate and time consuming to perform. Such methods are not therefore readily amenable to automation or to the analysis of large numbers of different samples.

A competitive PCR-based quantitation technique described by Cemu Bioteknik AB in WO 92/01812 represents an improvement over such techniques, but nonetheless has drawbacks when it comes to the analysis of a large number of samples, for example in a diagnostic situation. This method is based on the technique described in WO 90/11369 for the detection of immobilized amplified nucleic acids (designated DIANA) and involves competitive titration wherein amounts of target DNA are co-amplified with differing, known amounts of competitor DNA to produce different ratios of target: competitor DNA, the competitor DNA being substantially the same as the target DNA except that is comprises a recognition site which may be detected directly or indirectly by a labelled species. Different known amounts of the competitor DNA are added to aliquots of the sample generally as a series of equally stepped dilutions. A set of readings corresponding to the label values in each aliquot is obtained which, when plotted against the known amounts of added competitor DNA give a characteristic sigmoid curve; the point of inflection on the curve is defined by the sharp change in the amount of labelled DNA between those aliquots in which added competitor DNA predominated and those in which target DNA predominated and is approximately proportional to the amount of target DNA in the initial sample.

Whilst the so-called “quantitative DIANA” technique of WO 92/01812 avoids the use of disadvantageous quantification methods inherent in the other prior art methods mentioned above, it requires that a large number of determinations (and hence PCR reactions) be performed, in order to determine one unknown. Thus for example, for one sample at least 8 different measurement points are in practice required, in the form of a dilution series of the competitor DNA. The technique is therefore not only costly, in terms of the large number of reactions required, but requires large initial sample volumes. This renders the method unsuited to the analysis of large numbers of samples, such as occurs in a clinical diagnostic laboratory.

Various other competitive PCR techniques used to generate quantitative data are known. Competitive PCR is based on the addition to the sample of a known amount of a competitor molecule (typically DNA or RNA) which acts as an internal standard. This competitor molecule has the same primer recognition sites as the target gene and thus is expected to behave in the same way during PCR cycling.

The next step is the method of discrimination between target and competitor and the technique used to obtain information regarding how much of each molecule is present after amplification. In competitive PCR (where the amount of competitor in absolute terms is known) the absolute determination of the signals corresponding to the target and the competitor is not required. The final quantitation of the target is simply derived from the ratio between target and competitor. A common method for discrimination between target and competitor is through generation of amplicons which differ in length. Separation is thus achieved by gel-electrophoresis and ethidium bromide staining of the bands. Radioactively or fluorescence-labelled dNTPs can provide a more sensitive procedure than ethidium bromide staining. However amplicon length based discrimination is not ideal as the length of the template molecule may affect amplification efficiency.

A PCR product detection method taking advantage of the 5′ exonuclease activity of Taq DNA polymerase and dual-labelled fluorescent probes has also been described (Heid, C. A. et al. [1996] Genome Research 6, 986-94). In the event of successful amplification, the detection probe hybridises to the target/competitor and thereby it is accessible to the 5′→3′ exonucleases activity of the Taq DNA polymerase. The detection probe is non-extendable and incorporates a fluorescent reporter dye and a quencher dye. Due to the closeness of quencher to reporter, the reporter fluorescence is suppressed until cleavage of the probe occurs during PCR cycling. This is a rather expensive technique and there is a need for a more cost-effective and technically simple assay.

It has now been found that a simple and reliable method for obtaining qualitative and quantitative data about nucleic acid in a sample, which allows high throughput, can be performed using an internal standard where discrimination is achieved by detection of a primer (chain) extension reaction. Whether or not an extension reaction takes place on a given template will be dependent upon the normal rules of base pairing, either at the extension stage and/or at extension primer hybridisation. The method disclosed herein is particularly suited to automation e.g. in systems where the washing and injection steps take place in a microtitre plate format. The methods are particularly suitable for monitoring virus copy number.

Accordingly, the present invention provides a method of assessing the amount of target nucleic acid in a sample which comprises co-amplifying the target nucleic acid and at least one competitor nucleic acid molecule wherein each competitor molecule contains a unique discriminatory sequence, and determining the relative amounts of the respective amplicons, characterised in that determination is achieved by detecting a primer extension reaction using each said amplicon as template.

Analysis of the data obtained by the above method may be performed immediately or at a later time by the person performing the above determination or by others. Thus, according to the invention is also provided a method of assessing the amount of target nucleic acid in a sample which comprises co-amplifying the target nucleic acid and at least one competitor nucleic acid molecule wherein each competitor molecule contains a unique discriminatory sequence, and determining the relative amounts of the respective amplicons, wherein the information about the relative amounts of the respective amplicons is correlated to provide a value for the amount of target nucleic acid in the sample, characterised in that determination is achieved by detecting a primer extension reaction using each said amplicon as template.

Conveniently, the target nucleic acid may be DNA, although quantitation of target RNA (e.g. mRNA)is also within the scope of the invention. If it is desired to assess the amount of RNA in a sample, the method will additionally include the step of generating cDNA from RNA prior to co-amplification. Such synthesis can be carried out in an initial treatment step with a reverse transcriptase, conveniently in the same system of buffers and bases to be used in the subsequent amplification. Since the amplification procedures require heating to effect strand separation, the reverse transcriptase may be inactivated in the first amplification cycle. The enzymatic activity of both a reverse transcriptase and a polymerase which is stable may thus be used which conveniently may take the form of an enzyme with both activities, for example the polymerase. When MRNA is the target nucleic acid, it may be advantageous to submit the initial sample, e.g. a serum sample, to treatment with an immobilized poly dT oligonucleotide in order to retrieve all mRNA via the terminal poly A sequences thereof. Alternatively, a specific oligonucleotide sequence may be used to retrieve the RNA via a specific RNA sequence. The oligonucleotide can then serve as a primer for cDNA synthesis, as described in WO 90/06044.

It will be appreciated that whilst the target RNA may first be reverse transcribed into cDNA prior to amplification in the presence of one or more competitor DNA molecules, more conveniently, one or more competitor RNA molecules may be introduced at the stage of reverse transcription such that cDNAs of the target and competitor RNA are produced which may then be amplified.

The sample may be any sample containing genetic material, and all biological and clinical samples are included i.e. any cell or tissue sample of an organism, or any body fluid or preparation derived therefrom, as well as samples such as cell cultures, cell preparations, cell lysates etc. Environmental samples, e.g. soil and water samples or food samples are also included. The samples may be freshly prepared or they may be prior-treated in any convenient way e.g. for storage. The target nucleic acid may thus be derived or obtained from all such sources of nucleic acid material.

Representative samples thus include any material containing nucleic acid, including for example foods and allied products, clinical and environmental samples. However, the sample will generally be a biological sample, which may contain any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue-green algae, fungi, bacteria, protozoa etc. Representative samples thus include whole blood and blood-derived products such as plasma, serum and buffy coat, urine, faeces, cerebrospinal fluid or any other body fluids, tissues, cell cultures, cell suspensions etc. The sample may be pre-treated in any convenient or desired way to prepare the nucleic acid molecule for use (i.e. reaction) in the method of the invention, for example by cell lysis or purification, isolation, copying or pre-amplification of the nucleic acid etc.

The term “co-amplifying” includes any method for in vitro amplification which can be used to amplify both the target nucleic acid and competitor sequences. In this regard, both linear and exponential amplification methods may be used. As will be described further below, any convenient or desired method of in vitro amplification may be used, according to techniques well known and described in the art. The competitor molecules may conveniently be added to the sample, or to aliquots of the sample. Conveniently, the target and competitor molecules are amplified using the same amplification primers.

The term “unique discriminatory sequence” refers to a region of the competitor nucleic acid molecule which differs both from the equivalent region in the target molecule and the equivalent region in any other competitor molecule present in the amplification reaction. Thus, each competitor will comprise a region of sequence variation, wherein this region (i.e. the “sequence”) is different from the target and different from other competitors. The “sequence” comprises at least one base, preferably more than one, more preferably more than two bases. Suitable sequences for use in the methods of the invention comprise 2-10 bases, typically 3-10, 3-8 or 3-6, preferably 3-5, more preferably 3 or 4 bases. Thus, the unique discriminatory sequence may be the above ranges in length i.e. 1 to 10 bases, or 3-10, 3-8, 3-6, 3-5, 3 or 4 bases etc.

Preferably the discriminatory sequence will be a homopolymer, e.g. 2 or more or 3 or more, e.g. 3-6, 2-6, or more preferably 3 or 4 or 5 adjacent identical bases. The discriminatory sequences will typically be predetermined and the competitor molecule will have been designed to incorporate the unique discriminatory sequence but preferably be substantially the same as or identical to the target sequence and any other competitor molecule apart from in the region of the discriminatory sequence. The competitor molecule may conveniently be linear or added to the sample as a plasmid.

The amplicons derived from the target and competitor nucleic acid molecules will conveniently incorporate the unique discriminatory sequence or the equivalent region in the target molecule. Apart from the discriminatory sequence, the amplicons derived from the competitor molecule will be substantially the same as the target molecule and any other competitor molecule present. The amplicons will be substantially the same apart from the unique discriminatory sequence. By ‘substantially the same’ is meant the sequences have at least a 70%, preferably at least a 75% or 80% more preferably at least a 90, 95 or 98% sequence identity, as determined using the Fasta search (Pearson and Lipman (1988), Proc. Natl. Acad. Sci. USA 5: 2444-2448) as part of the GCG packages using default values; word size: 6; Gap creation penalty: 12.00; Gap extension penalty: 4.0, and constant Pam factor. The amplicons can also be regarded as ‘substantially the same’ if the amplicon derived from the competitor molecule is capable of hybridising to the complement of the amplicon derived from the target molecule under high stringency conditions. Conditions of high stringency may readily be determined according to techniques well known in the art, as described for example in Sambrook et al., 1989, Molecular Cloning, A Laboratory Manual, 2nd Edition. Hybridising sequences included within the scope of the invention are those binding under non-stringent conditions (6×SC/50% formamide at room temperature) and washed under conditions of high stringency (e.g. 2×SSC, 65° C.), where SSC=0.15 M NaCl, 0.015M sodium citrate, pH 7.2. Generally speaking, the aim is for the target and competitors to be as similar as possible, (aside from the discriminatory region) and consequently, it is preferred for variation between the target and competitors to be minimised outside the discriminatory region, and in particular for the variation to be confined to the discriminatory region. Thus, it is preferred for the target and competitor to be substantially similar in the region which is to be amplified. In other words, it is preferred that the competitor and target molecules are identical in sequence, except in the discriminatory region. However, it must be accepted that minor variation may occur, for example due to base misincorporation etc.

The amplicons derived from competitor and target molecules are preferably substantially the same length, i.e. differ in length by less than 30 bases, preferably less than 20 or 10 bases, most preferably are identical in length or are within 2 or 3 bases of the same length. Similarity of amplicons derived from the competitor molecules with each other and with the target in terms of length and C/G base composition is more important than similarity between the original starting competitor molecule and the target molecule.

Typically, before the method of the invention is performed a region of the target molecule will be identified, preferably one which provides 2 or more, e.g. 3 or 4 continuous identical bases. However the “region” may comprise just a single base. A competitor molecule will then be designed which is substantially the same as the target molecule (e.g. in the region to be amplified) except for the aforementioned identified region which in the competitor molecule provides the unique discriminatory sequence. The identified region in the target molecule will therefore be equivalent to the unique discriminatory sequence in the competitor and is referred to as such herein.

Preferably, the competitor molecule contains a homopolymeric discriminatory sequence which is conveniently the same length as the homopolymeric region identified in the target but is made up of a different base. Further competitor molecules for use in the same method may be provided in an analogous way, incorporating a homopolymeric discriminatory sequence made up of a base other than those found in the identified region of the target molecule or any other competitor molecules. Thus, as nucleic acid typically comprises 4 different bases, there will advantageously be a target molecule and three types of competitor molecule, each type of competitor having a homopolymeric region made up of a different base.

In order to perform a primer extension reaction, it will be necessary to provide a primer for hybridisation to the amplicons. This “extension” primer may be one of the primers used in the amplification reaction but will preferably be a different primer. According to different versions of the method of the invention, the extension primer may be ‘generic’, i.e. the same primer is able to bind to the target nucleic acid and any amplicon derived from a competitor molecule. Such a generic primer may be one of the amplification primers. Advantageously, such a primer will bind substantially adjacent (e.g. within 6, preferably within 3 bases), preferably exactly adjacent to the unique discriminatory sequence of the competitor and the equivalent region of the target molecule. A primer extension reaction may then be performed dependent upon availability of nucleotides which can form a sequence which is complementary to the discriminatory sequence or the equivalent region in the target molecule.

Alternatively, different extension primers are provided, each being specific for either the target or a competitor molecule. Thus, these primers have a region which is complementary either to one of the unique discriminatory sequences of a competitor molecule or to the equivalent region in the target molecule. The specificity is of course achieved by virtue of complementary base pairing and, for all embodiments of the invention, primer design may be based on principles well known in the art. It is not necessary for the primer to have absolute complementarity to the discriminatory sequence or the equivalent region in the target molecule, but this may be preferred to improve specificity of binding. Such primers may be termed “match/mismatch primers”, wherein specificity for one of the substrates (a competitor or target molecule) means mismatch for the other substrate(s). The mismatch sequence is preferably located at the 3′ end of the primer In this embodiment of the invention, primer extension does not depend on the sequences adjacent to the primer but on the capability of an exonuclease-deficient DNA polymerase to distinguish between a match and a mismatch (at the 3′ termini) primer in an extension assay.

The ‘primer extension reaction’ according to the invention includes all forms of template-directed, polymerase-catalysed nucleic acid synthesis reactions. The primer extension reaction may only comprise the incorporation of one nucleotide, or may comprise the incorporation of 2, 3 or more nucleotides.

It will be understood that absolute specificity of primer-binding and extension cannot be guaranteed, and some tolerance must be allowed for, as in any biological system. Thus, references herein to specificity are intended to refer to substantial specificity e.g. of primer binding, such that the invention may nonetheless be performed.

Specificity of primer binding and extension may be improved by appropriate primer design and/or selection of conditions e.g. by designing primers with higher annealing temperatures to enable the primer annealing step to be carried out at higher more stringent temperatures.

Alternatively or additionally, the primer extension reaction may be performed at higher temperatures using a temperature-tolerant polymerase enzyme.

Conditions and reagents for primer extension reactions are well known in the art, and any of the standard methods and enzymes etc. may be used in this step (see e.g. Sambrook et al, (editors), Molecular Cloning: a laboratory manual (1989), Cold Spring Harbor Laboratory Press). Thus, the primer extension reaction is carried out in the presence of primer, deoxynucleotides and a suitable polymerase enzyme e.g. T7 polymerase, Klenow or Sequenase Ver 2.0 (USB USA), or indeed any suitable available polymerase enzyme. Conditions may be selected according to choice, having regard to procedures well known in the art.

According to the present invention, detection of a primer extension reaction can be performed in a number of ways, such as incorporation of labelled nucleotides (i.e. by detecting incorporation of a labelled nucleotide into the primer extension product, for example by detecting the amount of label in the primer extension reaction product) or by using labelled probes which are able to bind to the extended sequence. Alternatively a “TaqMan” probe may be used, which is a composite probe containing a reporter dye and quencher dye. (see Heid et al., supra). The additional TaqMan probe is annealed to the substrate (ssDNA) and extension may be monitored by the exonuclease activity of the polymerase, which degrades the TaqMan probe.

Preferably however, primer extension is detected by monitoring PPi release, preferably by luminometric detection.

PPi can be determined by many different methods and a number of enzymatic methods have been described in the literature (Reeves et al., (1969), Anal. Biochem., 28, 282-287; Guillory et al., (1971), Anal. Biochem., 39, 170-180; Johnson et al., (1968), Anal..Biochem., 15, 273; Cook et al., (1978), Anal. Biochem. 91, 557-565; and Drake et al., (1979), Anal. Biochem. 94, 117-120).

It is preferred to use luciferase and luciferin in combination to identify the release of pyrophosphate since the amount of light generated is substantially proportional to the amount of pyrophosphate released which, in turn, is directly proportional to the amount of base incorporated. The amount of light can readily be estimated by a suitable light sensitive device such as a luminometer.

Luciferin-luciferase reactions to detect the release of PPi are well known in the art. In particular, a method for continuous monitoring of PPi release based on the enzymes ATP sulphurylase and luciferase has been developed (Nyrén and Lundin, Anal. Biochem., 151, 504-509, 1985; Nyrén P., Enzymatic method for continuous monitoring of DNA polymerase activity (1987) Anal. Biochem Vol 167 (235-238)) and termed ELIDA (Enzymatic Luminometric Inorganic Pyrophosphate Detection Assay). The use of the enzymes luciferase and ATP sulphurylase, and particularly the use of the ELIDA method to detect PPi is preferred according to the present invention. The method may however be modified, for example by the use of a more thermostable luciferase (Kaliyama et al., 1994, Biosci. Biotech. Biochem., 58, 1170-1171) and/or ATP sulfurylase (Onda et al., 1996, Bioscience, Biotechnology and Biochemistry, 60:10, 1740-42). This method is based on the following reactions:

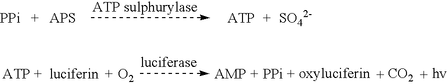

Reference may also be made to WO 98/13523 and WO 98/28448, which, whilst directed to pyrophosphate detection-based sequencing procedures, disclose PPi detection methods which may be of use in the present invention.

A potential problem which has previously been observed with PPi-based sequencing methods is that DATP, used in the chain extension reaction, (or any other adenine nucleotide used in the primer extension reaction such as dideoxy ATP (ddATP)) interferes in the subsequent luciferase-based detection reaction by acting as a substrate for the luciferase enzyme. This may be reduced or avoided by using, in place of deoxyadenosine triphosphate (ATP) (i.e. in place of the adenine nucleotide), a dATP (or other adenine nucleotide) analogue which is capable of acting as a substrate for a polymerase but incapable of acting as a substrate for a PPi-detection enzyme.

The term “incapable of acting” includes also analogues which are poor substrates for the detection enzymes, or which are substantially incapable of acting as substrates, such that there is substantially no, negligible, or no significant interference in the PPi detection reaction.

Thus, a further preferred feature of the invention is the use of a DATP analogue which does not interfere in the enzymatic PPi detection reaction but which nonetheless may be normally incorporated into a growing DNA chain by a polymerase. By “normally incorporated” is meant that the nucleotide is incorporated with normal, proper base pairing. In the preferred embodiment of the invention where luciferase is a PPi detection enzyme, the preferred analogue for use according to the invention is an α-thio analogue, e.g. the [1-thio]triphosphate (or α-thiotriphosphate) analogue of deoxy ATP, preferably deoxyadenosine [1-thio]triphospate, or deoxyadenosine α-thiotriphosphate (dATPαS) as it is also known. dATPαS, along with the α-thio analogues of dCTP, dGTP and dTTP, may be purchased from New England Nuclear Labs. Experiments have shown that substituting dATP with dATPαS allows efficient incorporation by the polymerase with a low background signal due to the absence of an interaction between dATPαS and luciferase. The signal-to-noise ratio is increased by using a nucleotide analogue in place of dATP, which eliminates the background caused by the ability of DATP to function as a substrate for luciferase. In particular, an efficient incorporation with the polymerase may be achieved while the background signal due to the generation of light by the luciferin-luciferase system resulting from DATP interference is substantially decreased. The α-thio analogues of other nucleotides (e.g. dNTPαS analogues of the other nucleotides) may also be used in place of all nucleotides (e.g. dNTPs).

The pyrophosphate detection step results in a signal indicative of the amount of pyrophosphate released. It has been confirmed that this signal is proportional to the amount of template present i.e. amplicons derived from target or competitor and therefore the signal is proportional to the number of target and competitor molecules originally present. If the amount of competitor is known, it follows that the amount of target can be calculated. Preferably, a calibration curve based on detection of extension reactions from competitor templates is generated and this can be used to read off quantitative information regarding the amount of target nucleic acid present in the sample. There is a linear correlation between the signal (i.e. the amount of PPi released) and the relative amount of template.

The term “target nucleic acid” is intended to encompass inter alia DNA, cDNA e.g. from retroviral RNA, genomic DNA, mitochondrial DNA, RNA, mRNA and PNA. The DNA may be single or double stranded.

The term “complementary” as used herein is intended to encompass any nucleic acid molecule which is complementary to the nucleic acid in question, its complementary sequence or its RNA or DNA equivalent or complementary sequence thereof. Whilst absolute complementarity is preferred, less than absolute complementarity, for example due to the occasional base misincorporation may be tolerated. In general, it is accepted for the complementarity to be “substantial”, within the limits and definitions given above in relation to “substantial identity” i.e. with the % homology and hybridisation conditions and limits given above.

The term “competitor nucleic acid” as used herein is intended to encompass any piece of DNA (or RNA after reverse transcription or PNA) which would compete with the target DNA (or RNA after reverse transcription or PNA) for binding to at least one of the primers used in the amplification reaction. “Competitor nucleic acid” extends also to the use of chimers of RNA, DNA and/or PNA. It will be appreciated however that it is an essential requirement of the method that all the nucleic acid sequences which are amplified in the amplification reaction must be amplified at a comparable rate and must therefore not be restricted with regard to the availability of essential reagents, ie. primers. Thus although referred to as competitor nucleic acid, this nucleic acid would only compete with the target nucleic acid when limiting concentrations of primers were available. For performance of the invention, this would not be the case. An excess of primers is generally used. The competitor may be single or double stranded.

The term “assessing” as used herein includes both quantitation in the sense of obtaining an absolute value for the amount of target nucleic acid in a sample, and also obtaining a semi-quantitative assessment or other indication, eg an index or ratio, of the amount of target nucleic acid in the sample. Thus, for example a ratio of the amount of target to competitor molecule may be obtained. The competitor molecule(s) is(are) subjected to the co-amplification step in a known amount (e.g. a known amount or concentration of the competitor may be added to the target nucleic acid or to the sample). Since the amount of competitor is known, it may be used to assess the amount of target.

More particularly, “assessing” is used herein to refer to obtaining a quantitative assessment of the amount of target nucleic acid in the sample by virtue of a comparison of the amount of target nucleic acid present in a sample with a known amount of a competitor. Such a comparison is allowed due to measurement of a primer extension reaction where both target and competitor provide/act as templates. As discussed herein, the invention provides methods whereby extension of a primer hybridised to an amplicon derived from a target molecule can be differentiated from extension of a primer hybridised to an amplicon derived from a competitor molecule. Differentiation may be achieved through specificity of extension primer hybridisation or through the extension reaction itself, for example by the ability to bring about an extension reaction when nucleotide availability is restricted. For example, there will only be a primer extension reaction when the extension primer is annealed adjacent to the discriminatory sequence if a nucleotide for incorporation is supplied which forms a base pair with the nucleotide adjacent to the 3′ end of the extension primer.

As mentioned above, the method of the invention may comprise the step of correlating the relative amounts of the respective amplicons (i.e. as obtained in the co-amplification step) to determine, or assess, the amount of target nucleic acid in the sample. As discussed above, the “relative amounts” may be an absolute assessment of the amounts of the respective amplicons (and hence of the respective amounts of the target and competitor molecules present (e.g. in the sample)). Alternatively, a ratio of the respective amounts may be obtained, or some other index or indication which may provide an index, or indication, or value for the amount of target nucleic acid in the sample. The “correlation” step may be performed simply by comparing the respective amounts determined or assessed, or by any other technique or calculation known in the art, for example by plotting a curve, comparing with a standard curve etc.

As mentioned above, the method requires that the competitor and target nucleic acids are amplified to the same extent, i.e. substantially at the same rate or with the same or substantially the same amplification efficiency, albeit to different levels as a result of the presence of different concentrations in the starting reaction. It will thus be appreciated that it is advantageous to ensure that the size and GC-content of the nucleic acids are kept substantially the same and that the sequence/s where the primer/s binds is identical. For this reason it is preferable to use competitor nucleic acids which have substantially the same sequence as the target nucleic acid; excluding the unique discriminatory region as discussed above.

Optionally, the competitor nucleic acid may be provided with a means for immobilization, which may be introduced during amplification, either through the nucleotide bases or the primer/s which is used to produce the amplified nucleic acid. The target nucleic acid may similarly be provided with a means for immobilization as a result of the amplification procedure. Preferably, therefore, one of the amplification primers will carry means for immobilization, or may be provided already immobilised on a solid support. Thus, the respective amplicons obtained may carry, or be provided with means for immobilisation or may be immobilised.

To facilitate immobilization, the primers used according to the invention may carry a means for immobilization either directly or indirectly. Thus, for example the primers may carry sequences which are complementary to sequences which can be attached directly or indirectly to an immobilizing support or may carry a moiety suitable for direct or indirect attachment to an immobilizing support through a binding partner.

Numerous suitable supports for immobilization of DNA and methods of attaching nucleotides to them, are well known-in the art and widely described in the literature. Thus for example, supports in the form of microtitre wells, tubes, dipsticks, particles, fibres or capillaries may be used, made for example of agarose, cellulose, alginate, teflon, latex or polystyrene. Advantageously, the support may comprise magnetic particles eg. the superparamagnetic beads produced by Dynal AS (Oslo, Norway) and sold under the trademark DYNABEADS. Chips may be used as solid supports to provide miniature experimental systems as described for example in Nilsson et al. (Anal. Biochem. (1995), 224:400-408).

The solid support may carry functional groups such as hydroxyl, carboxyl, aldehyde or amino groups for the attachment of the primer or capture oligonucleotide. These may in general be provided by treating the support to provide a surface coating of a polymer carrying one of such functional groups, eg. polyurethane together with a polyglycol to provide hydroxyl groups, or a cellulose derivative to provide hydroxyl groups, a polymer or copolymer of acrylic acid or methacrylic acid to provide carboxyl groups or an amino alkylated polymer to provide amino groups. U.S. Pat. No. 4,654,267 describes the introduction of many such surface coatings.

Alternatively, the support may carry other moieties for attachment, such as avidin or streptavidin (binding to biotin on the nucleotide sequence), DNA binding proteins (eg. the lac I repressor protein binding to a lac operator sequence which may be present in the primer or oligonucleotide), or antibodies or antibody fragments (binding to haptens eg. digoxigenin on the nucleotide sequence). The streptavidin/biotin binding system is very commonly used in molecular biology, due to the relative ease with which biotin can be incorporated within nucleotide sequences, and indeed the commercial availability of biotin-labelled nucleotides this represents one preferred method for attachment of. Streptavidin-coated DYNABEADS are commercially available from Dynal AS.

As mentioned above, immobilization may conveniently take place after amplification. To facilitate post amplification immobilisation, one or both of the amplification primers are provided with means for immobilization. Such means may comprise as discussed above, one of a pair of binding partners, which binds to the corresponding binding partner carried on the support. Suitable means for immobilization thus include biotin, haptens, or DNA sequences (such as the lac operator) binding to DNA binding proteins.

When immobilization of the amplification products is not performed, the products of the amplification reaction may simply be separated by for example, taking them up in a formamide solution (denaturing solution) and separating the products, for example by electrophoresis or by analysis using chip technology (mentioned hereinafter). Immobilization provides a ready and simple way to generate a single-stranded template for the extension reaction. As an alternative to immobilization, other methods may be used, for example asymmetric PCR, exonuclease protocols or quick denaturation/annealing protocols on double stranded templates may be used to generate single stranded DNA. Such techniques are well known in the art.

The method of the invention may be performed in a number of different ways. A number of particular specific embodiments are described further below, but generally speaking, the method may be performed using multiple competitor molecules or a single competitor molecule, and/or different amounts of the competitor molecules. Different primer extension reactions may be performed, (using the same or different extension primers, as mentioned above).

The principle is to perform a number of different primer extension reactions based on the competitor derived template(s), which may be selectively or specifically detected to generate signals (or calibration points) which may be correlated to the amount of template for each competitor or each primer extension reaction, and thus provide a basis for assessment of the amount of target nucleic acid in the sample (e.g. by comparison with the primer extension reaction(s) on a target-derived amplicon). Such a multiplicity of primer extension reactions may be performed by using a multiplicity of different competitor molecules, each used or present in a different amount, and performing a primer extension reaction on each different competitor amplicon, e.g. using either the same primer (and a template-specific or template-directed primer extension reaction, which as mentioned above may be accomplished by restricting the availability of nucleotides for incorporation or by using particular nucleotides) or by using different, template-specific extension primers, in order to discriminate or differentiate between the various competitor and target-based extension reactions. Alternatively, a single competitor may be used, but a multiplicity of different primer extension reactions may be performed, for example to generate primer extension products which may be distinguished, or to provide distinguishable primer extension signals, e.g. as discussed further below.

As used herein the terms “multiple” or “multiplicity” refer to 2 or more, e.g. 3 or more or 4, 5, 6 or more etc. e.g. 2 to 10, or more particularly 2 to 6, 2 to 5, 2 to 4, and preferably 3 or 4.

Where more than one competitor molecule is used, the concentrations of competitor nucleic acid should be selected to provide a standard curve (i.e. a calibration curve) in which the concentration of target nucleic acid falls within the range of the lowest and highest concentration of the competitor nucleic acid. Thus, for example, competitors at a concentration (or copy number) of 10×, 100× and 1000× would allow the determination of target nucleic acid at a concentration (or copy number) of 10× to 1000×. This range of concentrations can be used in low-copy applications such as HIV-1 using nested PCR or when the target is present at a high copy number/concentration such as expression analysis in which a single PCR run is sufficient to assess variations in expression levels.

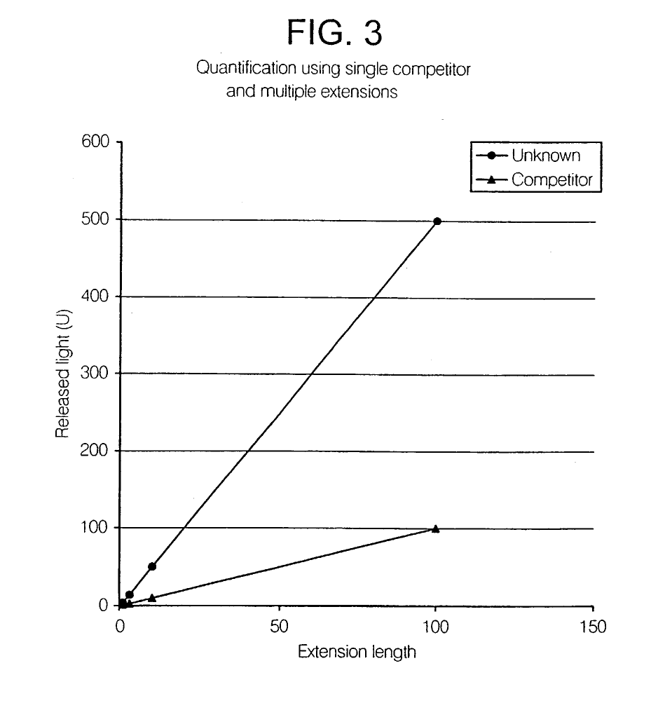

For the preparation of an internal calibration curve, at least two, preferably three different nucleic acid competitors at different concentrations should be used in the assay, unless the method shown in FIG. 3 is performed, which requires only one competitor concentration.

It will be appreciated that the sequence and length of the oligonucleotides to be used as amplification primers according to the invention will depend on the sequence of the target nucleic acid, the desired length of amplification product, the further functions of the primer (eg. means for immobilization) as well as the amplification procedure.

The in vitro amplification reaction may be any process which amplifies the nucleic acid present in the reaction under the direction of appropriate primers. The method may thus preferably be performed by PCR, and the various modifications thereof e.g. the use of nested primers, although it is not limited to this method. PCR will however generally be the method of choice. Those skilled in the art will appreciate that the invention would also be appropriate with amplification procedures such as Self-sustained Sequence Replication (3SR), NASBA, the Q-beta replicase amplification system and Ligase chain reaction (LCR) (see for example Abramson and Myers (1993) Current Opinion in Biotech., 4: 41-47).

Advantageously, to minimise any possible differences due to amplification efficiency, sufficient cycles of amplification are performed, to reach a saturation level (the so-called “plateau” phase of amplification). However, this is not essential and amplification may be performed in the exponential phase. If necessary, appropriate controls may be used to compensate for any differences in amplification, efficiency etc. The use of such controls is routine and widely known in the field of in vitro amplification.

For the determination of the number of copies (or concentration or amount) of target nucleic acid in the sample, the signals derived from primer extension of the amplified competitor nucleic acid may be used to generate a standard curve in which the level of signal is plotted against copy number (or concentration or amount) prior to amplification. Once the standard curve has been generated this can be used to read off the amount of starting copies (or concentration or amount) of target nucleic acid from the sample as reflected by the level of signal generated by primer extension of the amplified target nucleic acid. The “signal” may be luminescence when release of PPi is monitored but may derive from an incorporated label, e.g. a fluorescent or radio-label.

This method thus not only qualitatively positively identifies the presence of target sequence, unlike a number of the assays based on competition between target and competitor nucleic acid sequences for primers, but also allows the quantification of the amount of target nucleic acid present in the sample. Thus, the present invention provides a convenient single tube amplification protocol which allows quantification of PCR amplicons in a non-gel based bioluminometric assay.

Furthermore, by relying on a standard curve the method of the invention thus avoids the need for determination of the actual amount of amplified target DNA although this may in some cases be useful information.

Two-stage PCR (using nested primers), as described in WO90/11369, may be used to enhance the signal to noise ratio and thereby increase the sensitivity of the method according to the invention.

Regardless of whether one-stage or two stage PCR is performed, the efficiency of the PCR is not critical since the invention relies on amplification of competitor and target nucleic acid in the same reaction and thus all nucleic acid is amplified to the same extent.

The quantitative method according to the invention may be used for general quantification of RNA and DNA both for research and clinical applications, including diagnosis of viral, bacterial and protozoan pathogens. It may also find applications in forensic medicine, or environmental or contamination testing or monitoring.

Any suitable polymerase may be used, although it is preferred to use a thermophilic enzyme, such as Taq DNA polymerase, to permit the repeated temperature cycling without having to add further polymerase, e.g. Klenow fragment, in each cycle.

The method of the invention is very simple and rapid, thus making it easy to automate by using a robot apparatus where a large number of samples may be rapidly analysed. Since the preferred detection and quantification is based on a luminometric reaction, this can be easily followed spectrophotometrically. The use of luminometers is well known in the art and described in the literature.

The pyrophosphate-based nucleic acid quantification method of the present invention thus opens up the possibility for an automated approach for large-scale, non-electrophoretic analysis procedures, which allow for continuous measurement of the progress of the polymerisation reaction with time. The method of the invention also has the advantage that multiple samples may be handled in parallel.

The method may be adapted for use in various formats, for example in multi-welled microtitre plates or micro arrays (chip arrays) etc.

The method of the present invention is particularly advantageous in diagnosis of pathological conditions characterised by the presence of specific DNA, particularly latent infectious diseases such as viral infection by herpes, hepatitis, HIV or other viruses. Also, the method can be used with advantage to characterise or serotype and quantify bacterial, protozoal and fungal infections where samples of the infecting organism maybe difficult to obtain or where an isolated organism is difficult to grow in vitro for subsequent characterisation as in the case of P. falciparum or chlamydia species. Due to the simplicity and speed of the method it may also be used to detect other pathological agents which cause diseases such as gonorrhoea and syphilis. Even in cases where samples of the infecting organism may be easily obtained, the speed of the PCR technique compared with overnight incubation of a culture may make the method according to the invention preferable over conventional microbiological techniques.

The method of the present invention may be used in the detection of specific target RNA sequences. Thus, for example, the levels of RNA from retroviruses may be quantified. Alternatively, when present as a provirus, levels of target genomic viral DNA may be quantified. Subsequent references to viral RNA therefore include the possibility of assessing the levels of viral DNA. The method allows not only the positive identification of samples in which the target RNA is present, but also allow quantification of the levels of the target RNA. This has considerable clinical utility, for example in assessing the levels of virally infected patients over time, possibly during the course of treatment to establish the efficacy of a particular treatment or to establish the extent of infection.

It may also be possible to use quantitative data acquired from the method to determine the onset of viral infection by extrapolation with reference to the increasing levels of viral RNA in the same subject or analogous subjects. This may have significant implications in contagious diseases in which the identification of infection onset may allow the identification of other subjects which may be infected.

The method of the invention may be used for quantifying viral RNA or DNA e.g. HIV DNA or RNA, as a means of monitoring HIV infection. Thus viewed from a further aspect the present invention provides a method for assessing the amount of target viral (e.g. HIV) DNA or RNA in a sample from an infected patient using the aforementioned method.

The general method of the invention can be performed in a number of different ways and preferred embodiments of the method of the invention will now be described.

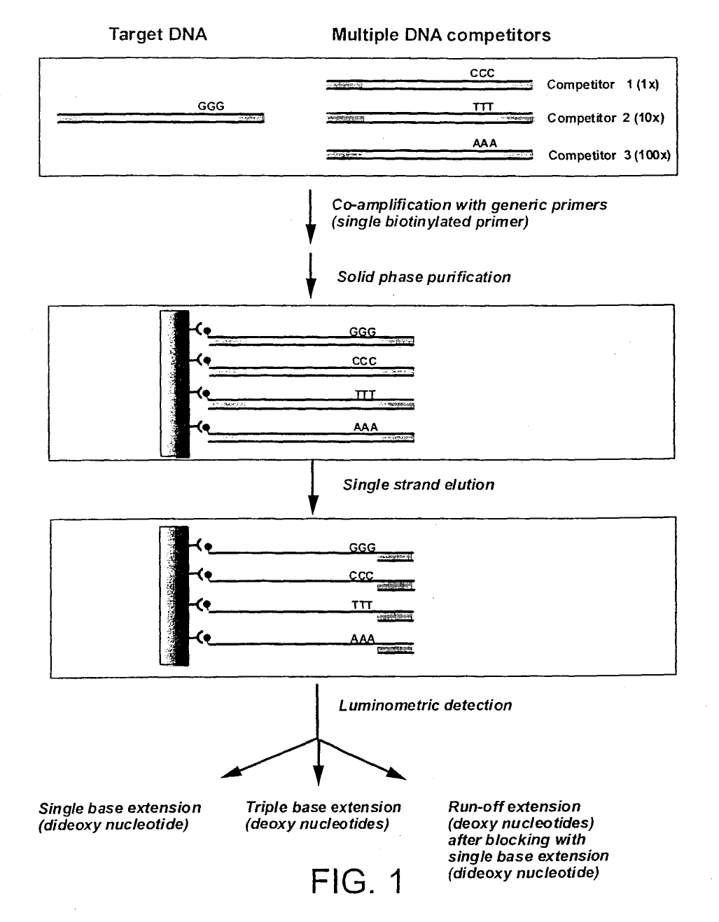

The first of these methods involves the use of more than one (i.e. multiple) type of competitor molecule, preferably more than 2 e.g. 3 different competitor molecules (see FIG. 1). Each competitor molecule differs from the target and the other competitor molecule(s) in its discriminatory sequence or equivalent region in the target molecule. The competitors are added to the sample containing the target nucleic acid and co-amplified. Preferably the primers used for amplification are biotinylated allowing immobilisation of the amplicons. The amplicons are conveniently rendered single stranded, e.g. by magnetic bead technology and elution. A generic extension (e.g. a sequencing) primer is annealed substantially adjacent or exactly adjacent to the unique discriminatory sequence of the competitors and the equivalent region of the target molecule.

Primer extension reactions to generate sequences complementary to part or all of the unique discriminatory sequence of the equivalent region in the target molecule can then be performed. Preferably, sequence extension is measured indirectly, as described above, by monitoring luminescence caused by pyrophosphate release. The sequence extension reactions may only involve incorporation of a single base; single base extension can be achieved by incorporation of dideoxy nucleotides which effectively “block” further extension.

Alternatively, and this is particularly convenient where the discriminatory sequences comprise a homopolymer of e.g. 2-6 e.g. 3 or 4 bases, two, three or four (or more etc.) base extensions can be performed on addition of deoxy nucleotides. For example, the deoxy nucleotide dCTP can be added, this will only result in a primer extension reaction with the template which incorporates a homopolymeric sequence of guanine bases. Such a template may either be derived from a competitor of known starting concentration/copy number or from the target nucleic acid.

As a third alternative, a “run off” extension can be performed. Here, after amplification, the sample may be divided into a number of aliquots depending on the number of different competitor molecules used. Then, to each sample dideoxy nucleotides are added, e.g. ddCTP and ddGTP or ddATP, ddTTP and ddGTP. This “blocks” all but one of the types of template molecule and means that only the template whose discriminatory sequence contains a base which forms a base pair with the base which has not been added as a dideoxy nucleotide, will undergo primer extension when deoxy nucleotides are added. Run off extension may go beyond the discriminatory sequence and the total signal for incorporation of e.g. 10, 20, 40, 80 or even 100 or 200 bases is obtained. A different template is allowed to undergo primer extension in each aliquot by appropriate use of dideoxy nucleotides.

In this way, a calibration curve can be obtained from either single base extension or homopolymeric base extension (typically of 2 to 4 bases) or run off extension. The ‘curve’ which is generally substantially linear will have multiple (i.e. 2 or more), preferably three points derived from the signal data from the competitors of known concentration/copy number. The value of the signal obtained from primer extension of the target templates-can then be used to read off a concentration/copy number for the amount of target nucleic acid present in the original sample.

Accordingly, in one preferred embodiment, the present invention provides a method of assessing the amount of target nucleic acid in a sample, which comprises

(i) co-amplifying the target nucleic acid together with multiple competitor nucleic acid molecules, wherein each said competitor molecule is different and comprises a unique discriminating sequence and wherein each said different competitor molecule is present in a different amount;

(ii) performing a primer extension reaction using each said respective amplicon obtained in step (i) as template, using the same extension primer for each respective template, wherein said primer extension reaction is template-specific; and

(iii) determining the amounts of the respective amplicons by detecting the results of each said primer extension reaction; and

(iv) assessing the amount of target nucleic acid in the sample from said amounts.

As mentioned above, the relative amounts of the respective amplicons from the target and from each of the competitor molecules may thus be determined, and these may be correlated to provide an assessment of the amount of target nucleic acid present in the sample.

By “template-specific” is meant that the primer extension reaction depends on the template, and may be detected in a template-specific manner. In other words, the different primer extension reactions on different templates may be discriminated or distinguished from one another. In this way individual or distinguishable “signals” or results may be obtained for each of the respective target or competitor derived templates. Thus, the primer extension reactions may be said to be directed, or more particularly template-directed, in the sense that different results are obtained for different templates. Such template-dependence may be accomplished by using particular nucleotides for incorporation in the primer extension step, or by restricting the availability of the nucleotides for incorporation.

The primer extension reactions may be performed in different ways; as discussed above, for example by single base extension or by multiple (i.e. 2 or more base extensions) e.g. extension by a few bases (for example by adding a nucleotide complementary to a homopolymeric discriminatory region) or run-off extension.

These individual (i.e. separate or distinguishable) results or signals may then be used to prepare a calibration curve from which the amount of target may be determined. In the co-amplification step, the different competitors are present in different amounts, which are known or predetermined. Thus, a known amount (or concentration etc.) of each competitor may be added to the target nucleic acid/sample (i.e. different amounts of the multiple competitors are co-amplified). Thus, the primer extension reactions specific for each amplicon will yield a different result, depending on the amount of template present (in turn dependent upon the amount of molecule originally present in or added to the sample). In other words, the “amount” (e.g. strength or intensity or duration) of signal may depend on the amount of template present, and since the “signals” from each template may be discriminated (and identified), a calibration curve of signal versus amount may be obtained. The bioluminometric PPi detection assay discussed above is particularly suited to detecting the primer extension reaction in this manner since it yields quantitative information (i.e. the amount of signal is dependent upon the amount of base incorporated, which in turn is dependent upon the amount of template present).

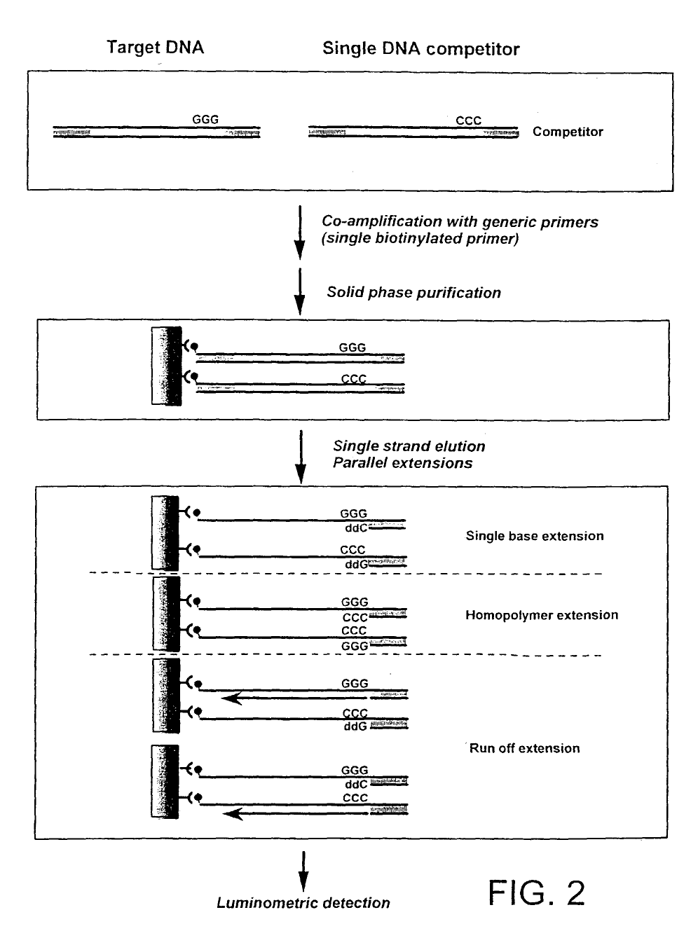

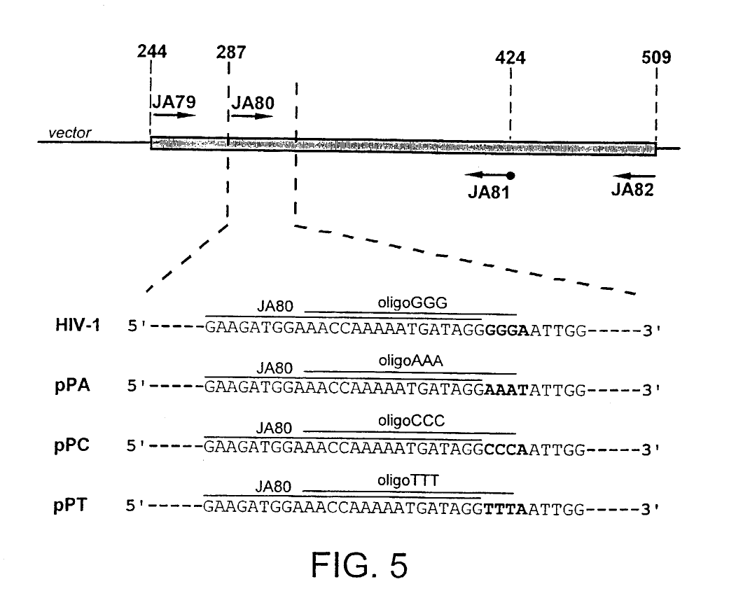

A variation of the above method is shown in FIG. 2. This method has the advantage that only a single species of competitor molecule is required. According to this method, multiple e.g. two or more, e.g. 3 or 4 or more, preferably 3 calibration points can be obtained by primer extensions of different lengths all based on the same competitor/primer complex. A competitor molecule of known concentration/copy number is amplified together with the target nucleic acid. After amplification, the competitor is analysed by two or more, e.g. three parallel extension reactions, e.g. single base extension using a dideoxy nucleotide, a homopolymeric extension of e.g. 3-10 base pairs using deoxy nucleotides and a run off extension e.g. 40-150 such as 80-120 preferably around 100 base pairs. The sequence formed by primer extension may include a sequence complementary to the discriminatory sequence or a part thereof. Alternatively, as discussed in more detail below, the extension primer may bind specifically to the discriminatory sequence as a match/mismatch primer. Differentiations involving match/mismatch primers is discussed below and shown in FIG. 5. Where the discriminatory sequence is more than one nucleotide in length, the extension primer may hybridise to part of the sequence and the extension reaction may involve synthesis of nucleic acid which is complementary to part of the discriminatory sequence. Thus primers will be specific for a competitor or target amplicon but extension will also be dependent on the bases in the discriminatory sequence and vary between competitor and target.

The datapoints can be plotted giving a linear slope (see FIG. 3) Table 1 below gives typical values for 4 extension reactions and these values have been used in FIG. 3 wherein the y-axis corresponds to the released light (i.e. amount of signal) and the x-axis corresponds to the extension length.

TABLE 1

Released light

| Extension length | Unknown | Competitor |
|---|---|---|
| 1 | 5 | 1 |
| 3 | 15 | 3 |
| 10 | 50 | 10 |
| 100 | 500 | 100 |

In a similar manner (adjusting the primer used and/or nucleotides added) the different extension are performed on templates derived from the target. Again these datapoints will generate a linear slope. The gradient of the slopes may then be used to calculate the absolute amount of target. For example, if the gradient of the slope for the competitor is 1 and this molecule was originally present in 100 copies, if the gradient at the slope of the target is 5 then this would have been present in the original sample in around 500 copies.

Accordingly, in a further preferred embodiment, the present invention provides a method of assessing the amount of target nucleic acid in a sample, which comprises

(i) co-amplifying the target nucleic acid and a known amount of a competitor nucleic acid molecule, wherein said competitor molecule comprises a unique discriminatory sequence, and is present in a known amount;

(ii) performing multiple primer extension reactions on each respective amplicon, wherein each said primer extension reaction yields an extension product of different length;

(iii) detecting the results of each said primer extension reaction, on each said respective amplicon; and

(iv) comparing the said results to determine the relative amount of the respective amplicons in order to provide an assessment of the amount of target nucleic acid in the sample.

The competitor and target nucleic acid molecules, and hence their respective amplicons, may be distinguished by virtue of the unique discriminatory sequence which differs in sequence from the corresponding or equivalent sequence in the target molecule.

The primer extension reactions on each respective template (i.e. competitor or target-derived) may be performed, as discussed above, using either the same extension primer or a different primer. In other words the extension primers may be (but need not be) specific for either the target or competitor amplicon.

Multiple primer extension reactions on each amplicon are designed to yield extension products of differing length. Such products may be distinguished when the multiple primer extension reactions are detected, and yield different detectable signals, which may be distinguished. Thus, each primer extension reaction will yield a separate “calibration point”, and a single competitor may thus yield multiple calibration points, the number of which depending on the number of different primer extension reactions performed. For example, the amount of signal may vary with the length of the extension produced. The length of extension product may thus be plotted against signal obtained, to generate a calibration curve. By comparing the respective results obtained for the competitor and target amplicons respectively, a correlation of the amount of target to the amount of competitor may be obtained. Since the amount of competitor is known, this enables the amount of target nucleic acid in the sample to be assessed.

The multiple primer extension reactions may be designed to generate of different length as described above e.g. single base extension, extension of a limited number of bases (e.g. 3 to 10 or 2 to 6) for example, by homopolymeric extension, or run-off extension.

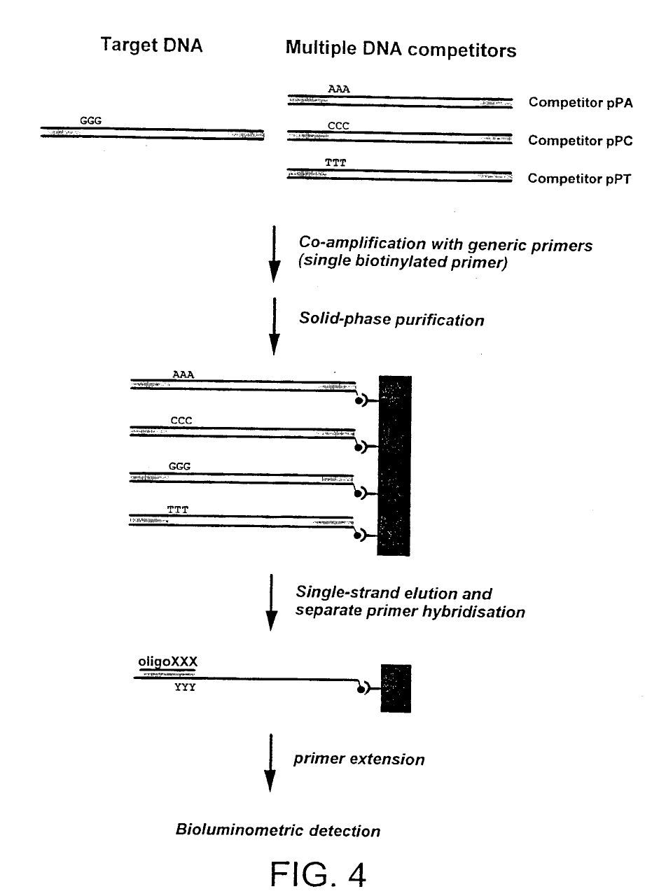

A further embodiment of the present invention is shown in FIG. 4. Here an extension primer binds specifically to part or all of a complementary discriminatory sequence of a competitor molecule or the equivalent region in the target molecule. Again multiple competitors of known concentrations are used but here detection of competitor and target is conveniently achieved using match/mismatch extension primers. The amplification mixture is conveniently immobilised and made single stranded before being divided into aliquots, each analysed with a different extension primer. Primers are designed with regions at their 3′ end which are specific for just one of the discriminatory sequences (or a part thereof) or the equivalent region of the target. Thus, the extension primers hybridise specifically to either the target amplicon or one of the competitor amplicons. The resulting complexes are used as substrates in primer extension assays to quantify the amount of each amplicon. The signals resulting from specific extension of each extension primer are directly correlated to the original amount of the target and competitors. The competitors can thereby be used to create an internal calibration curve in order to allow estimation of the amount of target present in the sample.

Preferably, the primer extension reaction based on each template will result in the sequences of the same length to facilitate comparison. This may conveniently be achieved by designing competitor molecules which are the same as the target molecule in the region which acts as template to be growing strand in the primer extension reaction. The reaction may result in incorporation of just a single nucleotide (e.g. by addition of dideoxynucleotides to the sample) or more than one nucleotide. The synthesised nucleic acid may comprise a homopolymeric sequence but as discrimination is primarily achieved by match/mismatch primers, all four deoxynucleotides can be added but significant primer extension will only occur when the extension primer complementary to the discriminatory sequence or equivalent region in the target is present.

Accordingly, in a still further preferred embodiment, the present invention provides a method of assessing the amount of target nucleic acid in a sample, which comprises

(i) co-amplifying the target nucleic acid together with multiple competitor nucleic acid molecules, wherein each said competitor molecule is different and comprises a unique discriminating sequence and wherein each said different competitor molecule is present in a different amount;

(ii) performing a primer extension reaction using each said respective amplicon obtained in step (i) as template using a different extension primer for each said respective template;

(iii) determining the amounts of the respective amplicons by detecting the results of each said primer extension reaction; and

(iv) assessing the amount of target nucleic acid in the sample from said amounts.

Thus, different extension primers are used, each specific for a particular template (i.e. the target amplicon or a particular competitor amplicon). Conveniently, as discussed above, the primers may be designed to be specific for the unique discriminatory region of each competitor, or for the equivalent or corresponding region in the target. Particularly advantageously, the primers may contain a match or mismatch for the discriminatory region at their 3′ end. In this manner, extension will only occur where there is a “match”. By detecting the respective extension reactions, signals may be obtained, which may be correlated to the amount of template (and hence amount of target/competitor) present, thereby allowing the amount of target nucleic acid in the sample to be assessed. In other words, as discussed for the first preferred embodiment as described above, the relative amounts of the respective amplicons from the target and from each of the competitor molecules may be determined and may be correlated to provide an assessment of the amount of target nucleic acid in the sample.

The invention also comprises kits for carrying out the method of the invention. These will normally include one or more of the following components:

one or more competitor nucleic acid molecules as defined above;

at least one primer for the primer extension reaction;

primer(s) for in vitro amplication;

nucleotides for amplication and/or for the primer extension reaction (as defined above);

a polymerase enzyme for the amplification and/or primer extension reaction; and

means for detecting primer extension (e.g. means of detecting pyrophosphate release as discussed and defined above).

Further optional components may include buffers, etc.

The invention will now be described by way of non-limiting examples with reference to the drawings in which:—

FIG. 1 shows schematically one method for the quantification of nucleic acid using multiple DNA competitors wherein a generic extension primer is annealed and three different extension reactions can be performed.

FIG. 2 shows schematically a further method for the quantification of nucleic acid using only a single competitor molecule, generic extension primers and a plurality of extension reactions.

FIG. 3 shows a typical calibration curve derived from the method represented in FIG. 2.

FIG. 4 shows schematically a method for the bioluminometric technique for quantification of nucleic acid. The mixture of amplicons resulting from the single-tube PCR is captured onto the solid phase and made single-stranded, before being divided into four aliquots and subjected to template-specific primer extension reactions. The inorganic pyrophosphate (PPi) released in the DNA polymerase-catalysed reaction is monitored by coupled enzymatic reactions using ATP sulfurylase and luciferase. Light generated as a result of a successful extension is measured by a luminometer.

FIG. 5 shows HIV-1 and competitor sequences. Nucleotides are numbered as described by Myers et al. The outer PCR (JA79-JA82) results in a 266 bp PCR product and the biotinylated, inner PCR product (JA80-JA81) is 138 bp. The bioluminometric detection using the three-bp 3′-end template-specific extension primers (oligoXXX) results in extension over 110 nucleotides.

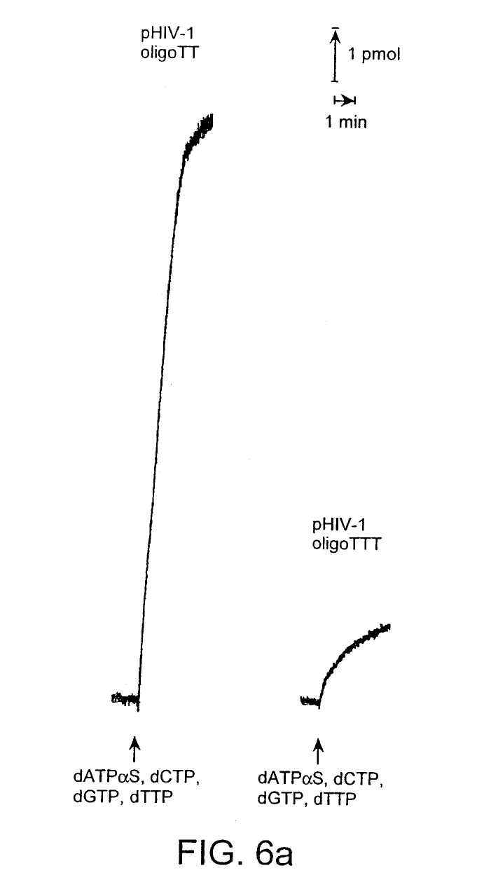

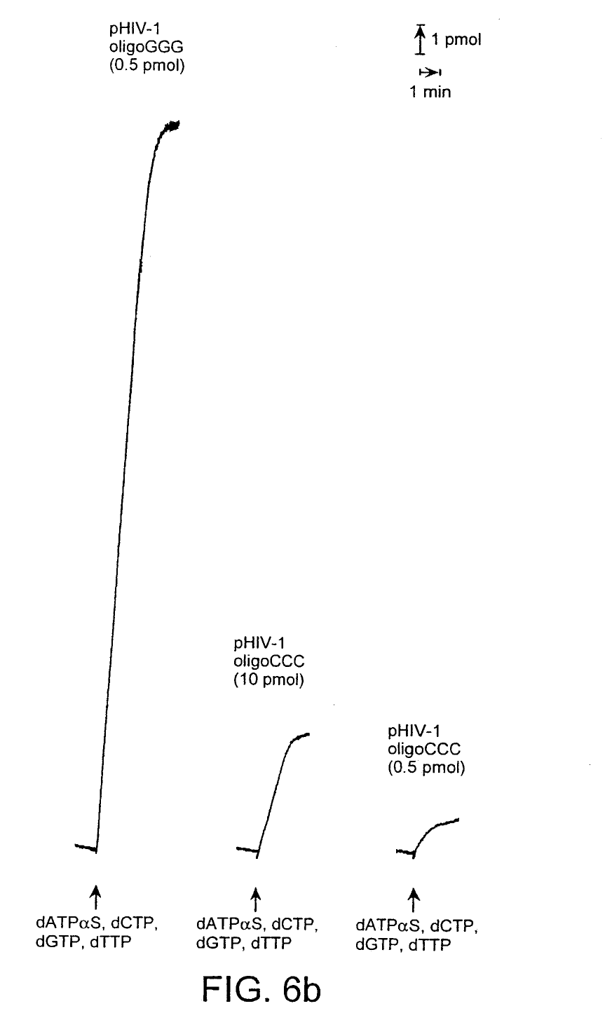

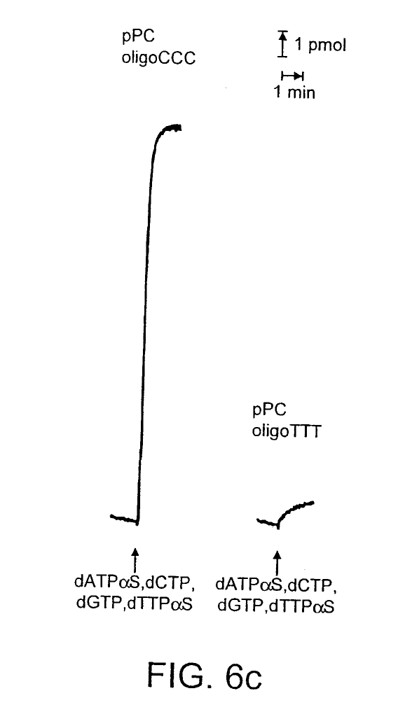

FIG. 6 shows typical traces from real-time match and mismatch primer extensions, obtained during optimisation of the bioluminometric primer extension assay. The reaction was started by addition of the indicated deoxynucleotides. (A): Comparison between extension primers with two and three 3′-end template-specific bases. (B): Mismatch extensions performed with 0.5 and 10 pmol of extension primer, respectively. (C): A representative example of background signal using the final conditions (see Example for details).

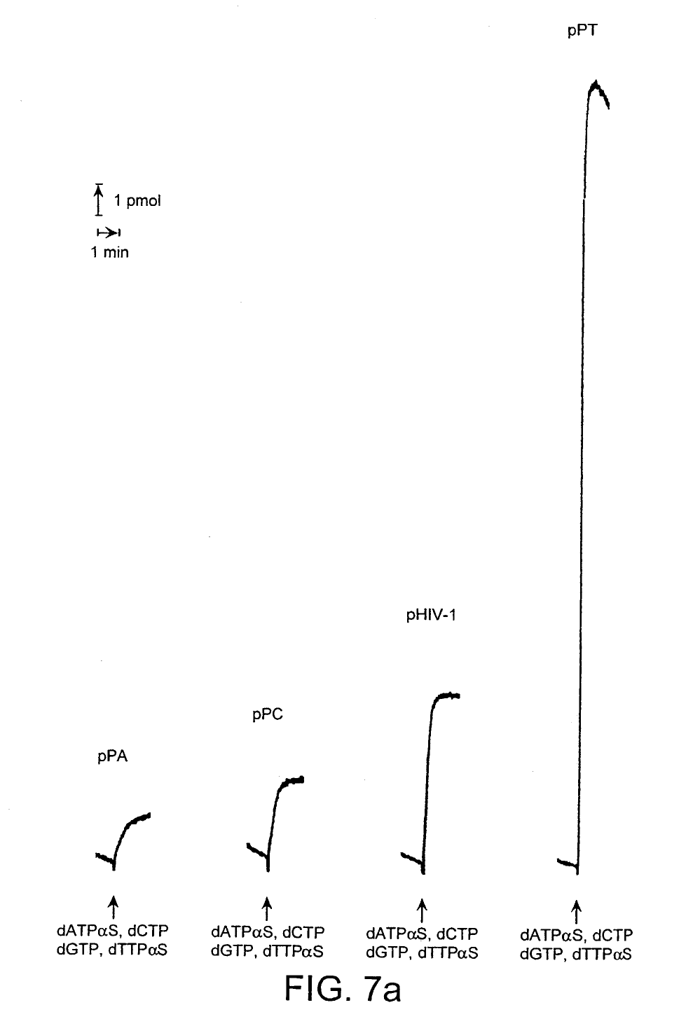

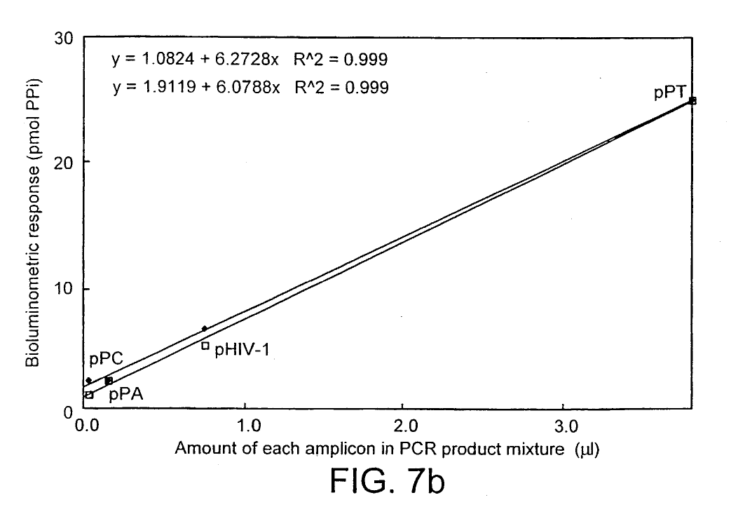

FIG. 7 shows luminometric traces (A) and calibration curve (B) from bioluminometric primer extension analysis of a mixture of PCR amplicons. The plasmids pPA, pPC, pHIV-1 and pPT were amplified separately and the PCR products were mixed in relative amounts 1:5:25:125. The mixture was then divided into four aliquots, each analysed with its respective extension primer. The different shapes of the bioluminescence curves reflect different enzyme kinetics at different concentrations of extension template.

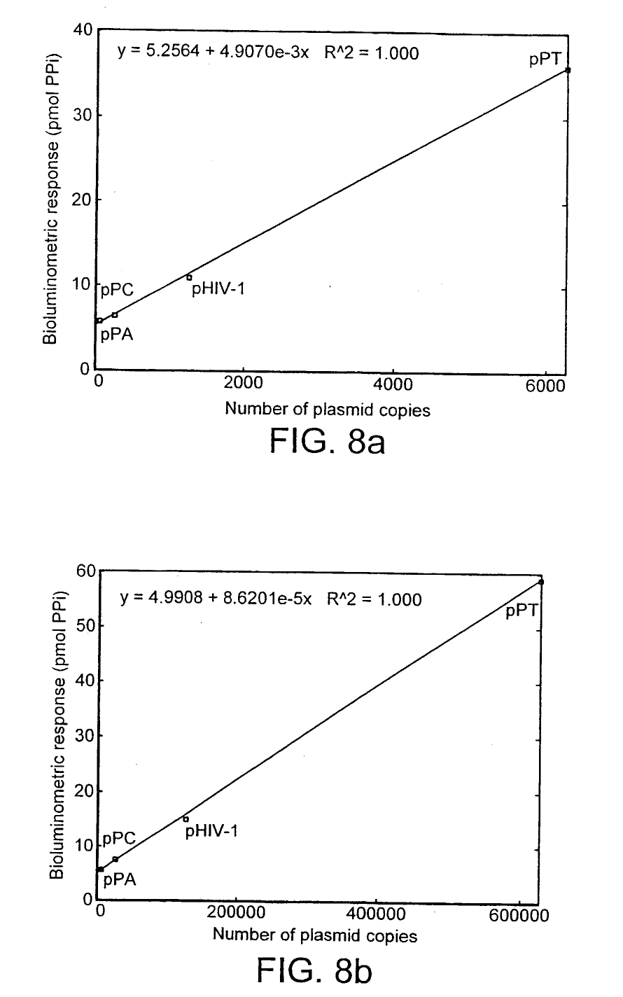

FIG. 8 shows calibration curves from bioluminometric analysis of experiments involving competitive PCR. Two mixtures of plasmids were analysed; one containing 50, 250, 1,250 and 6,250 copies of plasmids pPA, pPC, pHIV-1 and pPT, respectively (A) and one containing 5,000, 25,000, 125,000 and 625,000 copies (B).

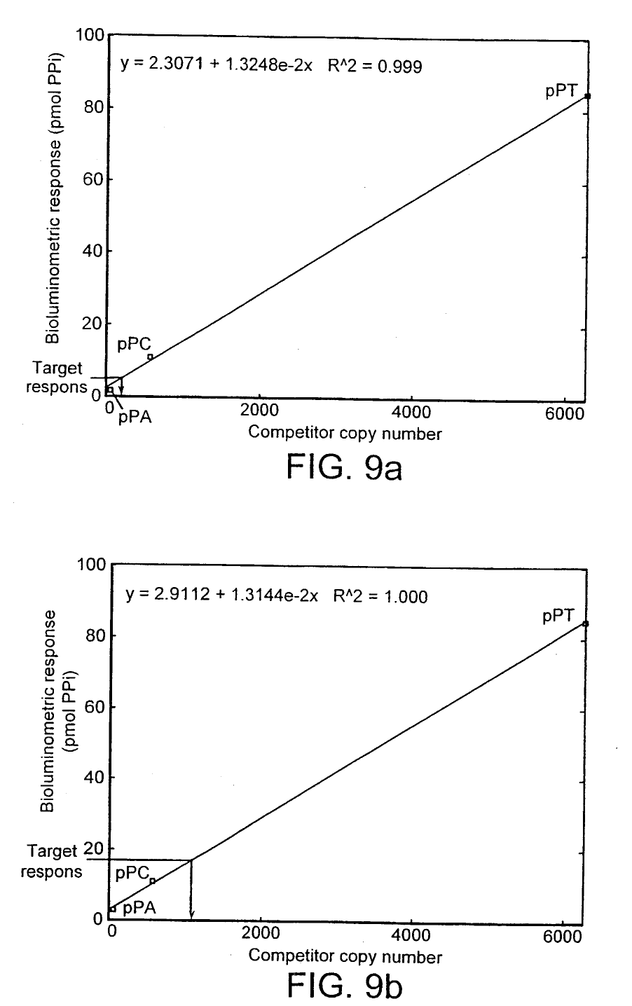

FIG. 9 shows calibration curves used for quantification of approximately 200 (A) and 1,000 (B) copies of MN strain HIV-1. The competitor mixture contained 50,560, and 6,250 copies of plasmids pPA, pPC and pPT, respectively.

### EXAMPLE 1

Preparation of MN Strain HIV-1 DNA

The model system described here used DNA from HIV-1 MN infected peripheral blood mononuclear cells (PBMC). These were diluted in crude cell lysates of uninfected PBMC to contain various numbers of viral HIV-1 copies. PBMC were isolated by Ficoll-Paque density centrifugation and lysed without prior cultivation in PCR lysis-buffer (10 mM Tris-HCl pH 8.3, 1 mM EDTA, 0.5% NP40, 0.5% Tween 20 and 300 mg/ml Proteinase K) at a concentration of1×10⁶ cells/100 μl as described previously (Wahlberg et al., (1991) AIDS Res Hum Retroviruses 7, 983-90). Crude cell lysates were used directly for PCR amplification.

Cloning of DNA Competitors

Three plasmids were constructed (pPA, pPC, pPT), containing nucleotides 244-509 of HIV-1 reverse transcriptase sequence (Myers et al., (1991) Human retroviruses and AIDS, Los Alamos National Library, Los Alamos) (FIG. 5). In these plasmids, a three or four bp substitution was introduced to enable discrimination between the wild-type HIV sequence and the three competitor sequences in the post-PCR detection system. A fourth plasmid (pHIV-1), containing wild-type HIV sequence, was also constructed. PCR cloning of competitors was achieved by mixing two partially overlapping PCR products covering the chosen region. Two ng of HIV-1 BH10 was used as template in the first reaction with JA79 and JA81 as primers, resulting in a 181 bp product. In the second reaction, resulting in a 223 bp product, the substitution primers PP1, PP2, PP3 and JA80, were each used together with JA82 to introduce the substitutions into the three competitors and to amplify the wild-type HIV-1 sequence. These primers are defined in Table 2 below.

TABLE 2
Oligonucleotides used

| | |
|---|---|
| PP1 | 5′-GAA GAT GGA AAC CAA AAA TGA TAG GCC CAA TTG GAG G-3′ |
| PP2 | 5′-GAA GAT GGA AAC CAA AAA TGA TAG GTT TAA TTG GAG G-3′ |
| PP3 | 5′-GAA GAT GGA AAC CAA AAA TGA TAG GAA ATA TTG GAG G-3′ |
| JA79 | 5′-ACA GGA GCA GAT GAT ACA GTA TTA G-3′ |
| JA80 | 5′-GAA GAT GGA AAC CAA AAA TGA TAG G-3′ |
| JA81 | 5′-biotin-CAA TTA TGT TGA CAG GTG TAG GTC C-3′ |
| JA82 | 5′-CCT GGC TTT AAT TTT ACT GGT ACA G-3′ |
| oligoAA | 5′-AAC CAA AAA TGA TAG GAA-3′ |
| oligoCC | 5′-AAC CAA AAA TGA TAG GCC-3′ |
| oligoGG | 5′-AAC CAA AAA TGA TAG GGG-3′ |
| oligoTT | 5′-AAC CAA AAA TGA TAG GTT-3′ |
| oligoAAA | 5′-AAC CAA AAA TGA TAG GAA A-3′ |
| oligoCCC | 5′-AAC CAA AAA TGA TAG GCC C-3′ |
| oligoGGG | 5′-AAC CAA AAA TGA TAG GGG G-3′ |
| oligoTTT | 5′-AAC CAA AAA TGA TAG GTT T-3′ |
| oligoCCC15 | 5′-AAA AAT GAT AGG CCC-3′ |

In the amplifications, 5 U of Pfu DNA polymerase (Stratagene Inc, La Jolla, Calif., USA) was used together with the recommended buffer, 0.2 mM of each dNTP and 0.2 μM of each primer. The PCR program consisted of initial denaturation at 95° C. for 5 min, 30 cycles of 95° C. for 30 s, 55° C. for 30 s and 72° C. for 2 min followed by 10 min of extension at 72° C. The PCR products were separated on a 2% agarose gel and purified using a QIAEX II kit (Qiagen GmbH, Hilden, Germany). The purified PCR products from the first and second reactions were mixed individually (to generate plasmids pPA, pPC, pHIV-1 and pPT) and used as template in a third PCR with the primers JA79 and JA82. AmpliTaq Gold DNA polymerase (Perkin-Elmer, Norwalk, Conn., USA) was used with the recommended buffer and 2 mM MgCl₂, 0.2 mM of each DNTP and 0.2 μM of each primer, which were added in the first PCR cycle.

The PCR program was as follows: 95° C. for 12 min, 68° C. for 12 min, 25 cycles of 95° C. for 30 s, 60° C. for 30 s, 72° C. for 1 min and finally 10 min of extension at 72° C. The four 266 bp products were purified using QIAEX II (Qiagen), ligated into AT cloning vector pGEM-T (Promega, Madison, Wis., USA) and transformed into Escherichia coli RR1ΔM15 (Ruther, U. (1982) Nucleic Acids Res 10, 5765-72). The resulting clones were PCR screened and sequenced (Hultman et al., (1989) Nucleic Acids Res 17, 4937-46). Correct clones of the four constructs were purified using Jetstar plasmid preparation kit (Genomed Inc., NC, USA). Plasmids were dissolved in 10 mM Tris-HCl (pH 8.3) and 2 mM EDTA and were analysed by agarose gel electrophoresis and by measurement of absorbance.

End-Point Dilution Analysis

Determination of plasmid concentrations was performed by using end-point dilution experiments involving nested PCR as outlined previously (Vener et al., (1996) Biotechniques 21, 248-52, 253-5). The plasmid preparations were diluted in PCR buffer II (Perkin Elmer) containing 10 ng/μl yeast RNA (Boehringer Mannheim, Mannheim, Germany). The numbers of DNA copies were calculated by the Poisson distribution formula (i.e. one starting copy corresponds to a dilution step in which 63% of the samples are positive by PCR (Brinchmann et al., (1991) J Virol 65, 2019-23)).

Competitive PCR

HIV-1 target and competitors were amplified with nested primers (see below) located in the polymerase gene of the HIV-1 genome. For the initial experiments, 1×10⁶ copies of the four plasmid constructs (containing the HIV wild-type sequence and the three competitor sequences, respectively) were amplified separately. During optimisation of the bioluminometric detection system, these PCR products were used pure or as a mixture of the four PCR products. In the experiments including competitive PCR, different configurations of plasmids were used as template. Mix A (50, 250, 1,250 and 6,250 copies of plasmids pPA, pPC, pHIV-1 and pPT respectively) and mix B (5,000, 25,000, 125,000 and 625,000 copies) were used to investigate whether the four plasmids were PCR amplified with the same efficiency. Mix C (50, 560 and 6,250 copies) was used for quantification of HIV-1 DNA.

The nested PCR was performed as follows: The templates (four plasmids or HIV-1 MN infected PBMC mixed with three competitors) were added to the outer PCR mixture resulting in final concentrations of 0.1 μM of each primer (JA 79 and JA 82), 50 μM of each dNTP, 1 U of AmpliTaq Gold DNA Polymerase, 10 mM Tris-HCl (pH 8.3), 50 mM KCl and 2.5 MM MgCl₂ in a reaction volume of 50 μl. The PCR program consisted of initial denaturation at 96° C. for 10 min, followed by a 30-cycle program consisting of 92° C. for 30 s, 50° C. for 30 s and 72° C. for 30 s, followed by extension at 72° C. for 1 min. In the inner amplification (primers JA80 and JA81), 2.5 μl of outer PCR product was used as template. The amplification conditions were the same as in the outer PCR except that 35 cycles of amplification was used.

Bioluminometric Quantification of PCR Products

To prepare the template for quantification, 5 μl of biotinylated PCR products were immobilised onto 300 μg streptavidin-coated super paramagnetic beads (Dynal AS, Oslo, Norway) as described by the manufacturer. Single-stranded DNA was obtained by removing the supernatant after incubation with 0.1 M NaOH for 5 min. The immobilised single-stranded DNA was washed once with 10 mM Tris-HCl (pH 7.5). Extension primers (0.5 pmol) oligoAAA, oligoCCC, oligoGGG and oligoTTT (Table 2) were then hybridised to the single-stranded DNA in 20 μl of buffer containing 0.1 M Tris-acetate (pH 7.75) and 20 mM MgAc₂ at 50° C. for 5 min after heating of the mixture at 95° C. for 1 min. The primed immobilised PCR products were washed once with binding/washing buffer (10 mM Tris-HCl (pH 7.5), 2 M NaCl, 1 mM EDTA, 0.1% Tween 20) and once with 0.1 M Tris-acetate (pH 7.75) buffer before addition of the 200 μstandard assay volume containing 0.1 M Tris-acetate (pH 7.75), 0.5 mM EDTA, 5 mM MgAc₂, 0.1% bovine serum albumin, 1 mM dithiothreitol, 5 μM adenosine 5′-phosphosulfate (APS), 80 μg polyvinylpyrrolidone (360,000), 20 μg D-luciferin (BioOrbit, Finland), 4 U of an exonuclease-deficient T7 DNA polymerase (Sequenase 2.0; US Biochemical, Cleveland, Ohio, USA), 30 mU of ATP sulfurylase (ATP:sulfate adenylyl transferase; EC 2.7.7.4) (Sigma Chemical Co., St Louis, Mo., USA), 40 ng of luciferase (Biothema, Sweden).

The reaction was started by the addition of 400 pmol of each dNTP, where deoxyadenosine-thiotriphosphate (dATPαS) and deoxythymidine-thiotriphosphate (dTTPαS) were substituted for the natural deoxynucleotides dATP and dTTP (Nyrén, P., Karamohamed et al., (1997) Anal Biochem 244, 367-73). The reaction was carried out at room temperature. The PPi released due to nucleotide incorporation was detected by the above-described system using a luminometer and a potentiometric recorder. The signal was measured 1 min after the reaction was started. The luminescence output was calibrated by the addition of a known amount of inorganic pyrophosphate (PPi).

Optimisation of the Bioluminometric Primer Extension Assay

During the initial optimisation experiments, template-specific extension primers differing by two nucleotides at their 3′-termini (oligoAA, oligoCC, oligoGG and oligoTT (Table 2)) were used. A shorter primer with three 3′ template-specific nucleotides (oligoCCClS (Table 2)) was also synthesised and compared to the nineteen-bp primer oligoCCC. The amount of extension primer was initially 10 pmol, but was in later experiments reduced to 0.5 pmol. Different combinations of the natural deoxynucleotides and α-thiotriphosphate deoxynucleosides were used in the primer extension assay to achieve a low background signal and a high polymerisation rate. Under suboptimal conditions, the reaction rate was slower and therefore the signal, intensity was measured after more than 1 min (i.e. when the match signal had reached the plateau phase).

### RESULTS

Principle of the Quantification Method

The principle of the competitive quantification method described above is outlined in FIG. 4. In this model system, the target (HIV-1 DNA) is co-amplified using nested primers with three plasmid DNA competitors designed to differ at only three or four adjacent nucleotide positions. The resulting biotinylated PCR fragments are immobilised onto streptavidin-coated paramagnetic beads. After alkali treatment, immobilised single-stranded DNA is divided into four aliquots, which are each mixed with one of four specific oligonucleotides. These extension primers hybridise specifically to either the wild-type HIV amplicon or to one of the three competitor amplicons. The four resulting complexes are used as substrates in separate primer extension assays to quantify the amount of each amplicon. The signals resulting from specific extension of each extension primer are directly correlated to the original amount of the target and competitors. The three competitors can thereby be used to create an internal calibration curve in order to allow estimation of the HIV target.

Construction of DNA Competitors

Three plasmid competitors (pPA, pPC and pPT) were designed and constructed by PCR cloning to contain mutated HIV-1 polymerase sequence (FIG. 5). PCR amplification of wild-type HIV-1 and the three competitors results in four amplicons of the same length (138 bp), but differing at the three or four nucleotides immediately downstream inner PCR primer JA80. The alterations were designed in a 3 bp homopolymeric stretch, i.e. the wild-type sequence comprising of three adjacent G nucleotides. The four mutated nucleotides enable discrimination between the amplicons using four specific extension primers in the primer extension assay. A plasmid containing HIV wild-type sequence (pHIV-1) was also constructed, to be used as target in the model system. The plasmid concentrations were determined by using limiting dilution analysis (Brinchmann et al., (1991) J Virol 65, 2019-23.). Mixtures of known amounts of the three competitors were then co-amplified with the wild-type HIV-1 target in a nested PCR.

Optimisation of the Bioluminometric Primer Extension Assay

Design and Specificity of Extension Primers

In order to evaluate the specificity of primer extension on the different constructed competitors, several “run-off” extension experiments were performed. The individual plasmid constructs were amplified and immobilised onto streptavidin-coated paramagnetic beads and rendered single-stranded by NaOH. Initially, these templates were investigated with four extension primers (oligoAA, oligoCC, oligoGG and oligoTT) designed to have two nucleotides at their 3′-ends complementary to only their respective target. When extension was performed on all combinations of extension primer and template, high background levels were obtained for some of the mismatch extensions. The combination resulting in the highest level of background was a two T:C (primer:template) mismatch (approximately 12% for extension primer oligoTT hybridised to competitor pHIV-1 (data not shown)).

Since the extension primer oligoTT was responsible for the highest level of background on all tested templates, an oligonucleotide with three template-specific bases at the 3′-end was synthesised (oligoTTT). When the new primer (oligoTTT) was compared to oligoTT in an experiment involving match and mismatch extensions, the background levels were reduced to approximately 13-37% (depending on target) of the background signals obtained with oligoTT. FIG. 6A shows the unspecific extensions by primers with two and three mismatches at the 3′-end on the template pHIV-1, for which the largest decrease in mismatch signal was observed. The arrow indicates the point of nucleotide addition and the accenting curve indicates the polymerase activity (in pmol of PPi produced) and time elapsed is indicated. In the assay, released pyrophosphate (PPi) is converted to ATP by ATP sulfurylase and the sequential production of light from ATP by luciferase is detected by a luminometer. Since DATP is a false substrate for luciferase, it was replaced by dATPαS which is silent for luciferase and efficiently incorporated by the DNA polymerase (Ronaghi M. et al (1996) Anal. Biochem 242, 84-9).

Use of Nucleotide Analogs

Another effort to reduce the mismatch extensions was to substitute the a-thiotriphosphate analogs (αS-dNTPs) for the remaining deoxynucleotides, since earlier studies have shown an improved discrimination between match and mismatch signals (one-bp mismatches (Nyrén et al., (1997) Anal Biochem 244, 367-73)). For some primer:template mismatch combinations (T:G and C:A) we observed a dramatic decrease in background, while the substitution was less effective in reducing the previously described background in extension of oligoTT hybridised to pHIV-1 (T:C mismatch). A drawback using α-thiotriphosphate analogs is the slower polymerisation rate in the match extensions, therefore, only the first correct bases after the 3′-mismatch termini (DATP and dTTP, see FIG. 5) were substituted and used together with the natural dCTP and dGTP in the final competitive PCR experiments.

Concentrations of Extension Primer

When performing extensions on all match and mismatch combinations using three-bp 3′ specific extension primers (oligoAAA, oligoCCC, oligoGGG and oligoTTT), oligoCCC hybridised to pHIV-1 (a C:C (primer:template) mismatch) surprisingly resulted in a background of approximately 11%, which was much higher than in the initial experiments, using the two-bases 3′ specific primer oligoCC. Since we believed that this background could be caused by mispriming rather than 3′ mismatch extension, the amount of extension primer was reduced from 10 to 0.5 pmol. This resulted in a background comparable to the other mismatch combinations (approximately 4% of the match signal) without affecting the corresponding match signal (FIG. 6B).

A four bases shorter primer (oligoCCC15) was synthesised and hybridised to pHIV-1 (0.5 and 10 pmol of primer, respectively). In the extension reactions where 10 pmol of primer was used in the hybridisation step, the shorter primer resulted in a considerably lower background, but for 0.5 pmol of primer no significant difference was observed.

Dynamic Range of the Bioluminometric Primer Extension Assay

The optimised protocol, in respect to mismatch extension and reaction rate was thus composed of the four extension primers with three alternating nucleotide stretches at their 3′-ends (oligoAAA, oligoCCC, oligoGGG and oligoTTT), specific for HIV-1 and the three competitors. To further increase the difference of primer extension rate of a match over a mismatch primer, the α-thio triphosphate analog for the first correct deoxynucleotide to be incorporated (DATP and dTTP) were used. All combinations of extension primer and PCR amplicon were analysed and all background signals were approximately 5% of the corresponding match signals. An example of the optimised system in which extension of competitor pPC amplicon hybridised to its corresponding extension primer oligoCCC is shown in FIG. 6C. The background signal from the combination of competitor pPC and extension primer oligoTTT i.e. a T:G (primer:template) mismatch is also demonstrated.

To further investigate the detection limit and the linearity of the bioluminometric primer extension assay, various amounts of one competitor amplicon (pPA) were detected in a background consisting of a mixture of the other three amplicons (pPC, pHIV-1 and pPT). It was possible to reproducibly detect 0.025 μl of pPA PCR product in a total of 5 μl PCR product, corresponding to a sensitivity of 1:200 (data not shown). In addition, a mixture of all four PCR amplicons (pPA, pPC, pHIV-1 and pPT in approximative relative amounts 1:5:25:125) was prepared, divided into four aliquots and analysed separately with all four extension primers. The obtained results demonstrate a linearity and conformity between duplicate samples (FIGS. 7A and B). The four different amplicons were also mixed in another configuration (125:25:5:1) and similar results were obtained (data not shown).

Competitive PCR on Mixtures of Four Plasmids

An important goal of the developed system was to allow for a single tube co-amplification of the multiple competitors requiring similar amplification efficiencies for the competitors and the target. Prior to co-amplification, the individual plasmid constructs were quantified by end-dilution series and nested PCR that give a statistical estimation of plasmid copy number (27, 35). Two different mixtures of all four plasmid templates were prepared; one low-copy number mixture containing 50, 250, 1,250 and 6,250 copies (mix A) and one high-copy number mixture containing 5,000, 25,000, 125,000 and 625,000 copies (mix B) of plasmids pPA, pPC, pPG and pPT, respectively. After nested PCR, the products were analysed by using the bioluminometric primer extension method. The results for the low-copy and high-copy mixtures (FIGS. 8A and B) show linearity in both intervals, thus indicating similar amplification efficiencies.

Quantification of HIV-1 MU Strain

To evaluate the quantification method on proviral HIV-1 DNA, a new competitor mixture containing 50, 560 and 6,250 copies (mix C) of plasmids pPA, pPC and pPT respectively was established. This mixture was then used with 200 and 1000 copies of HIV-1 MN DNA, also estimated by separate end-dilution experiments. Competitive PCR was followed by bioluminometric analysis as outlined above. The corresponding curves are shown in FIGS. 9A and B. A linear response was obtained making an estimation of the target copy number possible by using the internal calibration curves, which give an estimation of 203 and 1072 MN copies, respectively. The obtained results are in good agreement with the results obtained from end-point experiments.

1. A method of assessing the amount of target nucleic acid in a sample, which method comprises co-amplifying the target nucleic acid and at least one competitor nucleic acid molecule, wherein each competitor molecule contains a unique discriminatory sequence, said co-amplification yielding target- and competitor-derived amplicons respectively; performing a multiplicity of primer extension reactions using each said amplicon as template, wherein the various competitor- and target-based extension reactions are differentiated from one another; and determining the relative amounts of the respective amplicons, wherein said determination is achieved by detecting said primer extension reactions by luminometrically monitoring pyrophosphate release.

2. A method as claimed in claim 1, said method comprising correlating the relative amounts of the respective amplicons to assess the amount of target nucleic acid in the sample.

3. A method as claimed in claim 1 or claim 2, wherein multiple competitor molecules are used.

4. A method as claimed in claim 3 wherein different amounts of each said competitor molecule are used.

5. A method as claimed in claim 3 or claim 4, wherein primer extension reactions are performed on said respective amplicons using the same extension primer, wherein each said primer extension reaction is template-specific.

6. A method as claimed in claim 3 or claim 4, wherein primer extension reactions are performed on said respective amplicons using a different template-specific extension primer on each said template.

7. A method as claimed in claim 1 or claim 2, wherein a single competitor molecule is used.

8. A method as claimed in claim 7, wherein multiple primer extension reactions are performed on each said respective target- or competitor derived amplicon, wherein each said primer extension reaction yields an extension product of different length.

9. A method of assessing the amount of target nucleic acid in a sample, which comprises
(i) co-amplifying the target nucleic acid and a known amount of a competitor nucleic acid molecule, wherein said competitor molecule comprises a unique discriminatory sequence, and is present in a known amount;
(ii) performing multiple primer extension reactions on each respective amplicon, wherein each said primer extension reaction yields an extension product of different length;
(iii) detecting the results of each said primer extension reaction, on each said respective amplicon; and
(iv) comparing the said results to determine the relative amount of the respective amplicons in order to provide an assessment of the amount of target nucleic acid in the sample.

10. A method as claimed in claim 8 or claim 9, wherein 2 to 6 primer extension reactions are performed.

11. A method as claimed in any one of claims 7 to 10, wherein the primer extension reactions are performed using the same primer for the target- and competitor-derived amplicons.

12. A method as claimed in any one of claims 7 to 10, wherein the primer extension reactions are performed using -a different primer for the target- and competitor-derived amplicons.

13. A method of assessing the amount of target nucleic acid in a sample, which comprises
(i) co-amplifying the target nucleic acid together with multiple competitor nucleic acid molecules, wherein each said competitor molecule is different and comprises a unique discriminating sequence and wherein each said different competitor molecule is present in a different amount;
(ii) performing a primer extension reaction using each said respective amplicon obtained in step (i) as template, using the same extension primer for each respective template, wherein said primer extension reaction is template-specific; and
(iii) determining the amounts of the respective amplicons by selectively detecting the results of each said primer extension reaction, wherein each said primer extension reaction is differentiated from said other extension reactions; and
(iv) assessing the amount of target nucleic acid in the sample from said amounts.

14. A method of assessing the amount of target nucleic acid in a sample, which comprises
(i) co-amplifying the target nucleic acid together with multiple competitor nucleic acid molecules, wherein each said competitor molecule is different and comprises a unique discriminating sequence and wherein each said different competitor molecule is present in a different amount;
(ii) performing a primer extension reaction using each said respective amplicon obtained in step (i) as template using a different extension primer for each said respective template, wherein each said extension primer contains a match at its 3′ end for the discriminatory sequence of one of said competitor molecules or for the equivalent region in said target molecule;
(iii) determining the amounts of the respective amplicons by detecting the results of each said primer extension reaction; and
(iv) assessing the amount of target nucleic acid in the sample from said amounts.

15. A method as claimed in any one of claims 9 to 14, wherein the primer extension reactions are detected by detecting pyrophosphate (ppi) release.

16. A method as claimed in claim 15, wherein pyrophosphate (ppi) is detected luminometrically.

17. A method as claimed in any one of claims 1 to 8 or 16, wherein pyrophosphate is detected enzymically using the enzyme luciferase as a ppi-detection enzyme.

18. A method as claimed in claim 17, wherein in the primer extension reaction, an α-thio analogue of an adenine nucleotide is used.

19. A method as claimed in any one of claims 1 to 18, wherein said target and competitor nucleic acid molecules are co-amplified using amplification primers which are immobilised or carry means for immobilisation.

20. A method as claimed in any one of claims 3 to 6 or 13 to 19, wherein 2 to 6 competitor molecules are used.

21. A method as claimed in any one of claims 1 to 20, wherein said competitor molecule is identical to said target nucleic acid except in the region of the unique discriminatory sequence.

22. A method as claimed in any one of claims 1 to 21, wherein said discriminatory sequence comprises 1 to 10 bases.

23. A method as claimed in any one of claims 1 to 22, wherein said discriminatory sequence comprises 3 to 5 bases.

24. A method as claimed in any one of claims 1 to 23, wherein the discriminatory sequence is a homopolymeric region.

25. A kit for use in a method as defined in any one of claims 1 to 24, said kit comprising
(a) at least one competitor molecule as defined in any one of claims 1 to 24; and
(b) means for detecting a primer extension reaction.

26. A kit as claimed in claim 25, wherein said means is for luminometric detection of pyrophosphate release.

27. A kit as claimed in claim 25 or claim 26, further comprising one or more of the following components:
(c) at least one extension primer;
(d) primer(s) for in vitro amplication;
(e) a polymerase enzyme for the amplification and/or primer extension reaction.


SEQUENCE LISTING (17 sequences)

SEQ ID NO: 1 - DNA, 37 bp, Artificial
  misc_feature ()..(): PP1 artificial primer
gaagatggaaaccaaaaatgataggcccaattggagg

SEQ ID NO: 2 - DNA, 37 bp, Artificial
  misc_feature ()..(): PP2 artificial primer
gaagatggaaaccaaaaatgataggtttaattggagg

SEQ ID NO: 3 - DNA, 37 bp, Artificial
  misc_feature ()..(): PP3 artificial primer
gaagatggaaaccaaaaatgataggaaatattggagg

SEQ ID NO: 4 - DNA, 25 bp, Artificial
  misc_feature ()..(): JA79 artificial primer
acaggagcagatgatacagtattag

SEQ ID NO: 5 - DNA, 25 bp, Artificial
  misc_feature ()..(): JA80 artificial primer
gaagatggaaaccaaaaatgatagg

SEQ ID NO: 6 - DNA, 25 bp, Artificial
  misc_feature ()..(): JA81 artirficial primer
caattatgttgacaggtgtaggtcc

SEQ ID NO: 7 - DNA, 25 bp, Artificial
  misc_feature ()..(): JA82 artificial primer
cctggctttaattttactggtacag

SEQ ID NO: 8 - DNA, 18 bp, Artificial
  misc_feature ()..(): JA82 artificial primer
aaccaaaaatgataggaa

SEQ ID NO: 9 - DNA, 18 bp, Artificial
  misc_feature ()..(): oligAA artificial primer
aaccaaaaatgataggcc

SEQ ID NO: 10 - DNA, 18 bp, Artificial
  misc_feature ()..(): oligoCC artificial primer
aaccaaaaatgatagggg

SEQ ID NO: 11 - DNA, 18 bp, Artificial
  misc_feature ()..(): oligoGG artificial primer
aaccaaaaatgataggtt

SEQ ID NO: 12 - DNA, 19 bp, Artificial
  misc_feature ()..(): oligoTT artificial primer
aaccaaaaatgataggaaa

SEQ ID NO: 13 - DNA, 19 bp, Artificial
  misc_feature ()..(): oligoAAA artificial primer
aaccaaaaatgataggccc

SEQ ID NO: 14 - DNA, 19 bp, Artificial
  misc_feature ()..(): oligoCCC artificial primer
aaccaaaaatgataggggg

SEQ ID NO: 15 - DNA, 19 bp, Artificial
  misc_feature ()..(): oligoGGG artificial primer
aaccaaaaatgataggttt

SEQ ID NO: 16 - DNA, 15 bp, Artificial
  misc_feature ()..(): oligo CCC15 artificial  primer
aaaaatgataggccc

SEQ ID NO: 17 - DNA, 34 bp, Human immunodeficiency virus
gaagatggaaaccaaaaatgatagggggaattgg